(19) United States
(12) Patent Application Publication
(10) Pub. No.: US 2023/0329882 A1
(43) Pub. Date: Oct. 19, 2023

(54) ORTHOPEDIC PROSTHETIC DEVICE AND METHOD OF USE THEREIN

(71) Applicant: Thrive Orthopedics, LLC, Carmel, IN (US)

(72) Inventor: Kris KAIL, Carmel, IN (US)

(73) Assignee: Thrive Orthopedics, LLC, Carmel, IN (US)

(21) Appl. No.: 18/301,894

(22) Filed: Apr. 17, 2023

Related U.S. Application Data
(60) Provisional application No. 63/331,468, filed on Apr. 15, 2022.

(51) Int. Cl. A61F 2/50

(52) U.S. Cl. CPC A61F 2/5046; A61F 2002/5052; A61F 2002/6621

(57) ABSTRACT

An improved prosthesis and method for construction that utilizes at least a moldable material, footplate, and prosthetic portion to make a rapidly usable prosthetic specifically contoured to an interfacing region of an amputated appendage. The moldable material can generally be in the form of a multiple part material that is combined together to form a body safe and hardened material configured for placement on a specifically adapted surface, such as a footbed for placement within footwear.

### CROSS-REFERENCE TO RELATED APPLICATION

This application claims priority to U.S. Provisional Patent Application No. 63/331,468 filed 15 Apr. 2022, to the above-named inventor, and is herein incorporated by reference in its entirety.

### FIELD OF THE INVENTION

This present disclosure relates generally to a prosthetic device configured for creating and enabling better joint contouring at the site of an amputation and prosthetic component. In another aspect, the present disclosure relates to method of forming a prosthetic device to better contour to an amputation site of a patient to enable same day delivery of a prosthetic product.

### BACKGROUND

In the pedorthic, orthotic, prosthetic, and podiatric industries, it is known to create customized devices in the management and treatment of the foot and particularly amputations of the foot and/or toes. Generally, these devices are referred as toe prosthesis or partial foot prosthesis or as they are more commonly known as toe fillers. These devices are generally formed and constructed from universal materials, such as plastics, woods, foams, rubbers (natural and synthetic), and composites of these material types. Typically, production of these devices is outsourced to third parties that will mill, 3D print, or hand craft from a molded impression or images of the anatomical features and constructed by hand.

Accordingly, these customized devices can be time consuming to make, expensive, and may need to be further customized depending upon the type or style of footwear the device is used for. The third-party nature of fabrication also creates room for error in communication and device specifications. Additionally, as the patient is waiting for these customized devices, they are still having to utilize the damaged/amputate body part without or with an ill-fitting temporary device that can result in additional damage and/or infection to the amputation site. This can result in additional delays in healing and potentially additional adverse health effects.

Therefore, there exists a need for a better prosthetic device for placement within footwear to compensate for a missing toe(s) and a more efficient way to create these types of prosthetic devices, along with an improved method to create a more accurate contour of the prosthetic with the amputation site, as well as allow for same day fabrication of the prosthetic for the patient to utilize.

### BRIEF SUMMARY OF THE INVENTION

In one aspect, this disclosure is related to a prosthetic device that utilizes a series of critical interfaces that together streamline the manufacturing process and result in a more adjustable prosthetic. These interfaces can each be customized for a universal method that reduces the need for multiple sizes of kits. Customization of each interface can also be related to patient needs, such as offloading methods.

In another aspect, this disclosure is related to a prosthetic device that can utilize a moldable material that includes a moldable putty and activator combined together to form a body safe and hardened material configured for placement on a specifically adapted footbed for placement within footwear, in combination with a light weight section made of foam or a comparable material that can reduce weight in the forefoot and reduces the amount of putty material needed.

In another aspect, this disclosure is related to a prosthetic device that utilizes a moldable putty and activator combined together to form a body safe and hardened material configured for placement on a specifically adapted footbed for placement within footwear, shaped by using prefabricated, adjustable molds to shape the prosthetic device to the desired shape.

In another aspect, this disclosure is related to method and apparatus for creating a contoured prosthetic component to an amputation site that includes a generally in the form of a putty and activator combined together to form a body safe and hardened material configured for placement on a specifically adapted footbed for placement within footwear.

In another aspect, this disclosure is related to an improved method for constructing a customized and durable toe/partial foot prosthetic formed from multiple materials and/or layers in an assembled condition.

In another aspect, this disclosure is related to a prosthetic device that utilizes a putty in combination with a hollow form and footplate to form a durable and resilient prosthetic for use in footwear.

The invention now will be described more fully hereinafter with reference to the accompanying drawings, which are intended to be read in conjunction with both this summary, the detailed description and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

### BRIEF DESCRIPTION OF THE DRAWINGS

FIG. 1 is a cross-sectional side view of an exemplary embodiment of an amputation site filler prosthetic device of the present disclosure, particularly for a foot amputation.

FIG. 2 is a top view of an exemplary embodiment of an amputation site filler prosthetic device of the present disclosure, particularly for a foot amputation.

FIG. 3A is a side view of another exemplary embodiment of an amputation site filler prosthetic device and method of use of the present disclosure, particularly for a foot amputation.

FIG. 3B is a side view of another exemplary embodiment of an amputation site filler prosthetic device and method of use of the present disclosure, particularly for a foot amputation.

FIG. 3C is a side view of another exemplary embodiment of an amputation site filler prosthetic device and method of use of the present disclosure, particularly for a foot amputation.

FIG. 4A is a perspective exploded view of another exemplary embodiment of an amputation site filler prosthetic device and method of use of the present disclosure, particularly for a foot amputation utilizing a first type of forming enclosure.

FIG. 4B is a perspective exploded view of another exemplary embodiment of an amputation site filler prosthetic device and method of use of the present disclosure, particularly for a foot amputation utilizing a second type of forming enclosure.

FIG. 5A is a perspective view of an exemplary embodiment of a prosthetic device of the present disclosure.

FIG. 5B is a perspective view of an exemplary embodiment of a prosthetic portion of the present disclosure.

### DETAILED DESCRIPTION OF THE INVENTION

The following detailed description includes references to the accompanying drawings, which forms a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense.

Before the present invention of this disclosure is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the disclosure made herein.

Unless otherwise indicated, the words and phrases presented in this document have their ordinary meanings to one of skill in the art. Such ordinary meanings can be obtained by reference to their use in the art and by reference to general and scientific dictionaries.

References in the specification to “one embodiment” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The following explanations of certain terms are meant to be illustrative rather than exhaustive. These terms have their ordinary meanings given by usage in the art and in addition include the following explanations.

As used herein, the term “and/or” refers to any one of the items, any combination of the items, or all of the items with which this term is associated.

As used herein, the singular forms “a,” “an,” and “the” include plural reference unless the context clearly dictates otherwise.

As used herein, the terms “include,” “for example,” “such as,” and the like are used illustratively and are not intended to limit the present invention.

As used herein, the terms “preferred” and “preferably” refer to embodiments of the invention that may afford certain benefits, under certain circumstances. However, other embodiments may also be preferred, under the same or other circumstances.

Furthermore, the recitation of one or more preferred embodiments does not imply that other embodiments are not useful and is not intended to exclude other embodiments from the scope of the invention.

As used herein, the terms “front,” “back,” “rear,” “upper,” “lower,” “right,” and “left” in this description are merely used to identify the various elements as they are oriented in the FIGS, with “front,” “back,” and “rear” being relative to the apparatus. These terms are not meant to limit the elements that they describe, as the various elements may be oriented differently in various applications.

As used herein, the term “coupled” means the joining of two members directly or indirectly to one another. Such joining may be stationary in nature or movable in nature. Such joining may be achieved with the two members or the two members and any additional intermediate members being integrally formed as a single unitary body with one another or with the two members or the two members and any additional intermediate members being attached to one another. Such joining may be permanent in nature or alternatively may be removable or releasable in nature. Similarly, coupled can refer to a two member or elements being in communicatively coupled, wherein the two elements may be electronically, through various means, such as a metallic wire, wireless network, optical fiber, or other medium and methods.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element without departing from the teachings of the disclosure.

The present disclosure most generally relates to a material and method to provide a comfortable extension of a patient's residual limb that is less time consuming to manufacture when compared to the traditional solutions. Accordingly, the present disclosure most generally relates to prosthetic device that utilizes at least a moldable material 103 for use in the manufacture of a prosthetic device for placement within footwear. In an exemplary embodiment of the present disclosure, a two-part system comprises, but is not limited to, a putty, clay, or any other suitable moldable material that can be mixed together to form a cured substance. In one exemplary embodiment, the moldable material can be a siliconized mold putty provided in a pair of separate materials. Alternate to a siliconized mold putty, the moldable material 103 may be comprised of, but not limited to, a vinyl polysiloxane material that is provided in a pair of separate materials that is mixed together to form a cured substance. These siliconized based materials can be preferred for their material properties as the durometer (hardness/softness) of the material can be customized and utilized for different applications.

In some exemplary embodiments, the moldable material 103 can be beneficial for creating an intimate fit “socket” that creates total contact around the patients residual limb and contours to the amputated area of the residual limb. This total contact contour can better disperse pressure and reduce the risk of wound complications. The remaining aspects of the device can vary based on patient's needs. For example, under layer interfaces for cushioning or off-loading can be optionally included as needed on a case by case basis. Additionally, varying interfaces can be used in the forefoot to reduce the total weight of the prosthetic device based upon the desired use and type of prosthetic needed.

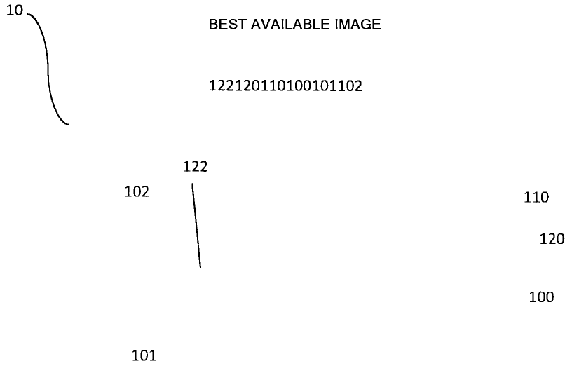

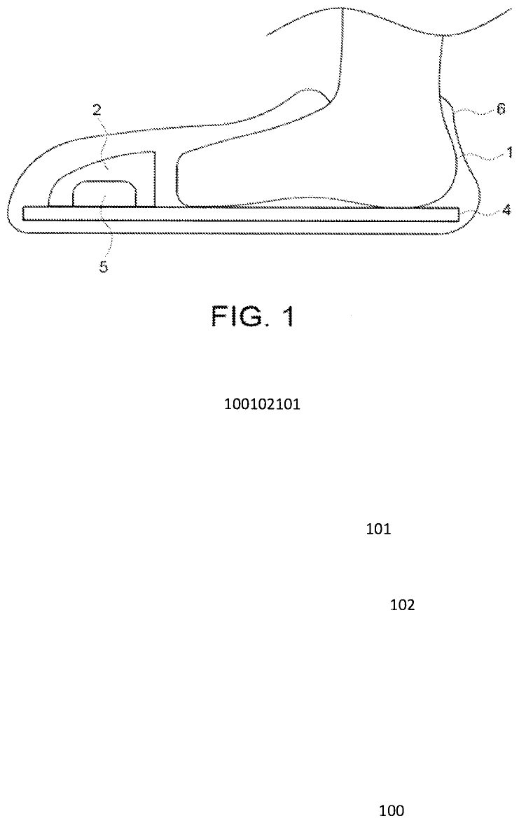

Referring now to FIGS. 1-5 of the prosthetic device and method for use of the present disclosure, generally referred to herein as device 10. The device 10 can best be understood as a device comprising various components and a method of construction for a prosthetic device utilizing these materials. More generally, the device 10 of the present disclosure can be utilized to generate a customized void filling contour that can be applied to different prosthetic and orthotic devices. The void filling materials 103 can then be married with existing devices and materials through coupling means including, but not limited to, adhesives, hardware, and common fasteners. In an exemplary use, for a patient having a transmetatarsal amputation, the device 10 can include a moldable material 103 that can be provided in a pair of components, that would be combined together to form an activated and cohesive material. This activated mixture of the moldable material 103 can then be shaped around the interfacing portion 122 of the residual part of the foot to create a “shoe filler” or “toe filler” that can be a customized shape configured to provide a functional benefit to the patient's gait cycle and reduce pressure and risk for skin breakdown on the residual part of the limb.

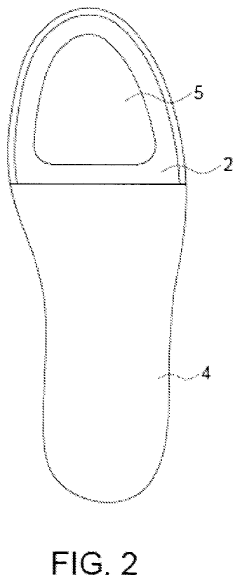

Referring now specifically to FIGS. 1-2, a side view and top side view of the device 10 components and method for use are shown in reference to a shoe interior 110. Accordingly, a the device 10 is constructed from the moldable material 103 can be mixed into a cohesive form that upon curing renders a final shape and can be secured to a footplate 100 having a size and shape to conform to the shoe interior 110. Preferably the footplate 100 can be constructed out of a carbon fiber material due to its weight, rigidity, and dynamic properties, although other strong materials, such as, but not limited to, steel, steel derivatives, plastics, or polymers may be utilized. When a large amount of the putty material is needed to fill a large space, a hollow component 101 of a mold cup or prosthetic cap component 102 may be used as a form to mold the moldable material 103 around. A hollow component 101 can reduce the amount and weight of the putty utilized in constructing the device 10. Accordingly, this hollow component 101 may be secured to the footplate 100 to generally reduce the volume of putty material needed for the device 10 during the customization process. In other exemplary embodiments, the prosthetic portion 102 can be a preformed shape that can later be trimmed to fit or contour within a defined space, such as a shoe. With the addition of the moldable material 103 and the footplate 100, any voids within a shoe 110 can be filled and generally result in reduced unwanted movement, which prevents ulceration of tissue of the foot 120.

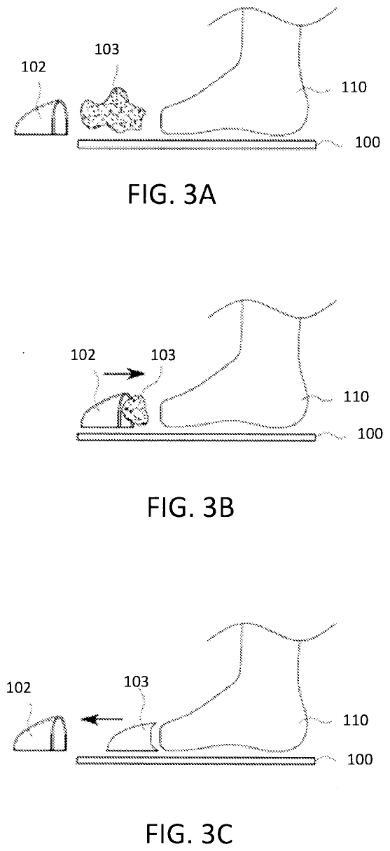

Referring now specifically to FIGS. 3A-C, an alternative device 10 and method for use is shown and illustrated. Accordingly, the device 10 can be constructed from the moldable material 103 through the use of a mold cup 102. In this method of assembly and customization, a foot 120 of the patient can first be placed onto a footplate 100, as shown in FIG. 3A, where the moldable material 103 and predefined mold cup 102 can then be positioned relative to the foot 120. As shown in FIG. 3B, two-part moldable material 103 mixture is combined and, prior to curing, is placed into the mold cup 102 for shaping, fitting, and contouring to the interfacing portion 122 of the amputated region of the patient. After the moldable material 103 is adequately contoured to the interfacing portion 122, the mold cup 102 can then be removed and the putty is allowed to cure and affixed to the footplate 100. Accordingly, the device 10 can utilize several shapes and sizes of mold cups 102 to aid in the shaping of the putty for a given type or style of footwear.

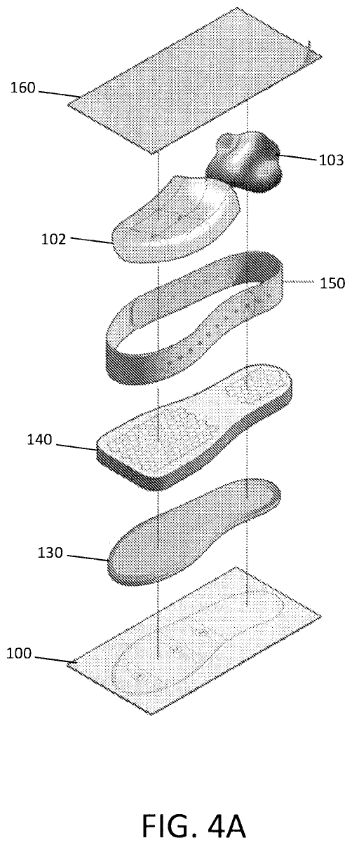

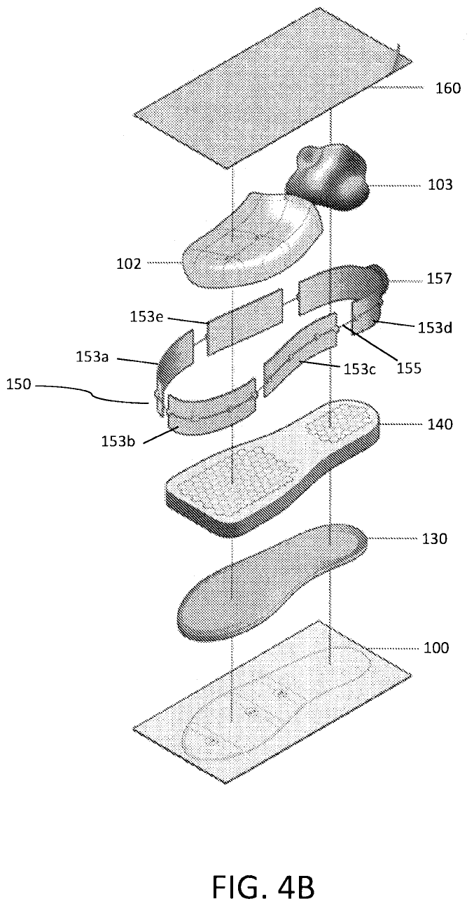

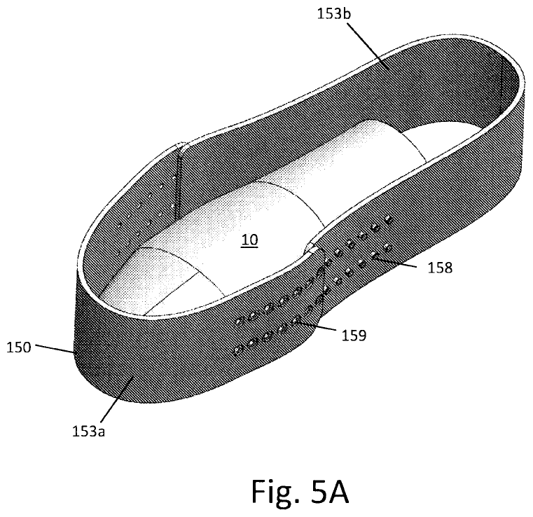

Referring now specifically to FIGS. 4A-B, an alternate exemplary embodiment of a device 10 and method of the present disclosure is shown. Accordingly, a base insole/footplate 100 can be cut from a foam or other suitable material based on the patients shoe size and shape. A patient/user's insole can be traced on the footplate 100 material and then cut out to the appropriate size and shape base for the insole of the patient. Optionally, additional layers for padding and/or offloading can also be shaped in a similar fashion. In some embodiments, the device 10 can include both a specific patient shoe insert 130 and additional insole component 140 (i.e. PegAssist™ or other insole components). In other various embodiments, the device may only include either the insole shoe insert 130 or the insole component 140.

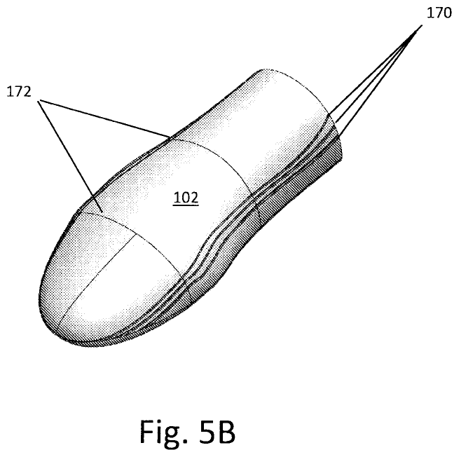

An enclosure system 150 can be utilized to help with forming around the exterior edge of the footplate 100 and the patient's foot 120. In some exemplary embodiments, the enclosure system 150 can be adjustable in nature to accommodate a wide variety of appendages sizes. In some embodiments, the enclosure system 150 can take the form of a snap together fastening means 152 that can couple a first portion 153a of the enclosure system to a second portion 153b as shown in FIG. 4A and FIG. 5A. In another exemplary embodiment, the enclosure system 150 can be comprised of a plurality of portions 153 that can be coupled together with a wire/cable 155. The cable can be coupled to a ratcheting/tensioning means 157 that can allow for the cable 155 to be retracted to decrease the outer diameter of the portions 153. In an exemplary embodiment shown in FIG. 5A, an enclosure system 150 comprised of a first portion 153a and a second portion 153b, the second portion can have one or more protrusions 158. The protrusions can be formed on each end of the second portion 153b. One or more corresponding apertures 159 can be formed on each end of the first portion 153a. It should be understood that the enclosure system could additionally be comprised of a single portion that has fastening means on each side of the portion.

In some exemplary embodiments, the prosthetic cap component 102 can be a universal appendage mold 102 that can be used to reduce weight in the forefoot. In one exemplary embodiment, appendage mold 102 can be a trimmable appendage mold 102 and composed from any suitable trimmable composition including but not limited to a foam material. A patient's appendage can be placed onto the footplate 100 and measured in relation to the appendage mold 102 to determine how much of the appendage mold needs to be trimmed in order to contour to the patient and/or to the apparatus which the mold 102 will be placed within, such as a shoe. In some exemplary embodiments, the mold 102 can have pre-defined standard cut guides 170 on the surface of the mold 102 as shown in FIG. 5C. The predefined marks 170 can be used as ordinary guides to account for typical foot sizes and common amputation sites. In some exemplary embodiments, the mold 102 can have pre-defined cut lines 170 along a longitudinal axis of the mold to account for shoe/foot width. Additionally or alternatively, the mold 102 can have pre-defined cut lines 172 along the latitudinal axis to account for shoe/foot length depending upon the user's needs. These cut marks can better allow a user to better form and trim the mold component 102 to conform to standard shoe sizes as well as reduce the amount of moldable material needed. Furthermore, the mold 102 can be lighter weight to provide better balance for the patient.

Once the mold 102 is trimmed and generally fills the majority of the negative space resulting from the amputation, the moldable material 103 can be placed and/or coupled on the back end of the mold 102 and then contoured to the contouring surface 122 of the patient's appendage. Once the moldable material 103 is contoured and fitted to a patient/user's residual limb 120, the enclosure system 150 can then be adjusted to provide an outer mold that can be used to contain the moldable material 103 to fit in the user's shoe while creating structure for the outer walls of the moldable material 103 as it is contoured to the interfacing portion 122 of the appendage. When a uniform and contoured joint between the moldable material 103 and the contouring surface 122 is achieved, the moldable material can be allowed to harden as well as fully coupled to the mold 102. In some exemplary embodiments, a fully formed prosthetic mold assembly can include both the mold 102 and the moldable material, which can then be coupled to the footplate 100. Alternatively, the fully formed prosthetic mold assembly can be coupled to a brace or any other suitable means to provide support for the user/patient.

The enclosure system 150 can be removed and additional material 160 can be optionally added to a top of the appendage mold 102 and the moldable material 103 to enclose and secure the interface between the mold 102 and the material 103. The additional material 160 can be any suitable material. In some exemplary embodiments, the additional material 160 can be a heat moldable film, film, canvas, or any other suitable material that can be coupled to the mold 102 and moldable material 103. In an exemplary embodiment, a padded film 160 having a pre-applied adhesive can be used to easily couple the various components together. The additional material 160 can allow for a final prosthetic component assembly to be formed while also providing additional padding for the user's anatomy. It should be understood that while this present disclosure primarily relates to molds for foot appendages, the method and apparatus of the present disclosure can be applied to other appendages as well.

While the invention has been described above in terms of specific embodiments, it is to be understood that the invention is not limited to these disclosed embodiments. Upon reading the teachings of this disclosure many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

What is claimed is:

1. A customizable prosthetic device, comprising:
a footplate; and
a contoured prosthetic cap assembly, comprising:
a prosthetic cap component, and
a moldable material component, wherein the moldable material component can be formed and contoured to an interfacing portion of an amputated appendage while resting on a top of the footplate, wherein the moldable material component can then be cured and fastened to the prosthetic cap component to form a customized prosthetic appendage, wherein the customize prosthetic appendage can then be fastened to the footplate.

2. The device of claim 1, further comprising an enclosure system configured to provide a lateral barrier around the customized prosthetic appendage when being contoured to the amputated appendage.

3. The device of claim 2, wherein the enclosure system is adjustable.

4. The device of claim 3, wherein the enclosure system comprises a first portion and a second portion, wherein the first portion is removably couplable from the second portion.

5. The device of claim 4, wherein the moldable material is provided in two parts that are mixed together to form the final moldable putty.

6. The device of claim 2, wherein the moldable material is comprised of a silicone-based material.

7. The device of claim 5, further comprising a wrapping material configured to be applied to the moldable material and the prosthetic cap component to couple the respective portion together.

8. The device of claim 7, wherein the wrapping material is a padded film with a pre-applied adhesive.

9. The device of claim 5, wherein the prosthetic cap component further includes at least one of the following:
pre-defined cut lines along a longitudinal axis of the cap component to account for shoe/foot width or pre-defined cut lines along the latitudinal axis.

10. A method of constructing a custom orthotic device for placement in an item of footwear of a user having an amputated appendage, the method comprising the steps of:
selecting a footplate sized for receipt within the item of footwear corresponding to the size of a foot of the user;
selecting a first moldable material component;
selecting a second moldable putty component;
mixing the first moldable putty component and the second moldable putty component together to form a moldable material;
forming the moldable material into a desired shape to contour to an interfacing region of an amputated appendage to generally function as a prosthetic device; and
securing the putty to the footplate.

11. The method of claim 10, wherein the moldable putty is comprised of a silicone-based material.

12. The method of claim 11, wherein the method includes the additional step of selecting a preformed prosthetic component for molding the moldable material around and coupling the preformed prosthetic component to the moldable material, wherein the preformed prosthetic component allows for the use of less moldable putty during construction, wherein the moldable material and prosthetic component can be coupled together to form a prosthetic assembly.

13. The method of claim 12, wherein the method includes the additional step of selecting a mold cup sized for the user and using the mold cup in forming the final shape of the cohesive moldable material.

14. The method of claim 13, wherein the prosthetic assembly is coupled to the footplate.

15. The method of claim 14, wherein the prosthetic component can include pre-defined cut lines along a longitudinal axis of the cap component to account for shoe/foot width and pre-defined cut lines along the latitudinal axis.

16. A customized orthotic device, the device comprising:
a moldable material capable of curing to a final shape; and
a footplate, wherein the moldable putty is configured for placement on the footplate to generally replace any anatomical features normally present on a footplate.

17. The customized orthotic device of claim 9, wherein the moldable putty material is provided in two parts that are mixed together to form the final moldable putty.

18. The customized orthotic device of claim 9, wherein the moldable putty is comprised of a silicone-based material.

19. The customized orthotic device of claim 9, wherein the footplate includes a hollow component for molding the moldable putty around, wherein the hollow component allows for the use of less moldable putty during construction.
